(19) United States
(12) Patent Application Publication
(10) Pub. No.: US 2026/0360539 A1
(43) Pub. Date: Nov. 12, 2026

(54) MENTAL ILLNESS PREDICTION METHOD, DEVICE, AND APPLICATION

(71) Applicant: Wistron Corporation, New Taipei City (TW)

(72) Inventors: PIN SHAN CHUANG, New Taipei City (TW); PEIYU HO, New Taipei City (TW)

(21) Appl. No.: 19/297,187

(22) Filed: Aug. 12, 2025

(30) Foreign Application Priority Data
May 7, 2025 (TW) 114117232

(51) Int. Cl. G16H 50/20; G06F 40/284; G16H 15/00; G16H 50/50

(52) U.S. Cl. CPC G16H 50/20; G06F 40/284; G16H 15/00; G16H 50/50

(57) ABSTRACT

Provided are mental illness prediction method, device, and application. The method includes: receiving user's speech and using a speech service application programming interface (API) to convert the user's speech into transcribed text; analyzing the transcribed text using a multi-layer analysis model to obtain a plurality of mental health indicators; and inferring the plurality of mental health indicators using a mental illness prediction model to predict a probability of mental illness occurrence. The device includes a mental illness prediction system, configured to predict the probability of mental illness occurrence, comprising: a speech processing module, an analysis module, and a prediction module. The application includes: transmitting the user's speech to the mental illness prediction system, and receiving the probability of mental illness occurrence returned by the mental illness prediction system.

### CROSS-REFERENCE TO RELATED APPLICATION

This non-provisional application claims priority under 35 U.S.C. § 119(a) to Patent Application No. 114117232 filed in Taiwan, R.O.C. on May 7, 2025, the entire contents of which are hereby incorporated by reference.

### BACKGROUND

### Technical Field

The present invention relates to a health assessment method, device and application, and in particular relates to a method, device and application for predicting the probability of dementia and depression occurrence by analyzing speech with an artificial intelligence model.

### Related Art

In today's society, people pay more and more attention to health issues, which indicates that there is an urgent need for more comprehensive and accurate health assessment methods. Traditional health check-ups primarily focus on physiological indicators, such as blood pressure and heartbeat. However, the assessment of mental health and cognitive ability is still relatively weak. In addition, with the intensification of the aging of the population, cognitive decline is becoming more and more serious. However, the early signs for cognitive decline are often subtle and difficult to detect, making early recognition and assessment quite challenging.

Current health assessment technologies still primarily rely on physiological indicators, and are supplemented by psychological questionnaires. However, these methods cannot fully grasp the mental state and cognitive function of a user, especially in the emotional expression and language features implied in language communication. In addition, existing early cognitive degradation assessment technologies are imperfect, and lack immediate support and intervention mechanisms. Traditional mental counseling and cognitive decline detection typically require professional involvement, thereby limiting their scope of application and convenience.

### SUMMARY

The present invention provides a natural language processing-based conversational health assessment system to provide more comprehensive health status assessment and support, thereby overcoming the deficiency of the prior art and achieving more accurate and thorough health assessment. By analyzing user conversations, emotional features, token choices, and intonations can be extracted from language to carry out emotional analysis and determination of the level of depression. Meanwhile, the signs of mental deterioration can also be detected through semantic coherence and language expression, so as to recognize depressive symptoms and cognitive decline at an early stage and provide appropriate support and intervention. Additionally, smartphones and cloud technology are combined for remote care, so that staff input errors and burdens can be reduced, and real-time health status assessments can be carried out during daily conversations of the user, thereby offering the user better health management solutions.

The present invention provides a mental illness prediction method, including: receiving user's speech and converting the user's speech into a transcribed text using a speech service application programming interface; analyzing the transcribed text using a multi-layer analysis model to obtain a plurality of mental health indicators; and inferring the plurality of mental health indicators using a mental illness prediction model to predict a probability of mental illness occurrence.

The present invention provides a mental illness prediction application, applied to a smart device, and including: a speech recording module, configured to record user's speech using the smart device; and a communication interface module, configured to transmit the user's speech to a mental illness prediction system, and receive a probability of mental illness occurrence that is returned by the mental illness prediction system.

The present invention provides a mental illness prediction device, configured for operation of a mental illness prediction system. The mental illness prediction system includes: a speech processing module, configured to receive user's speech and convert the user's speech into a transcribed text using a speech service application programming interface; an analysis module, configured to analyze the transcribed text using a multi-layer analysis model to obtain a plurality of mental health indicators; and a prediction module, configured to infer the plurality of mental health indicators using a mental illness prediction model to predict a probability of mental illness occurrence.

### BRIEF DESCRIPTION OF THE DRAWINGS

FIG. 1 illustrates a block diagram of a system architecture of a mental health assessment system according to an embodiment of the present invention;

FIG. 2 illustrates a block diagram of a system architecture of a mental illness prediction application according to an embodiment of the present invention;

FIG. 3 illustrates a block diagram of a first embodiment of a system architecture of a mental illness prediction system according to an embodiment of the present invention;

FIG. 4 illustrates a schematic diagram of multi-layer analysis model of a mental illness prediction system according to an embodiment of the present invention;

FIG. 5 illustrates a schematic diagram of mental health indicators of a mental illness prediction system according to an embodiment of the present invention;

FIG. 6 illustrates a schematic diagram of a second embodiment of a system architecture of a mental illness prediction system according to an embodiment of the present invention;

FIG. 7 illustrates a curve graph of mood scores of a depression mood determination model according to an embodiment of the present invention;

FIG. 8 illustrates a curve graph of cognitive scores of a dementia cognitive determination model according to an embodiment of the present invention; and

FIG. 9 illustrates a flowchart of a mental illness prediction method according to an embodiment of the present invention.

### DETAILED DESCRIPTION

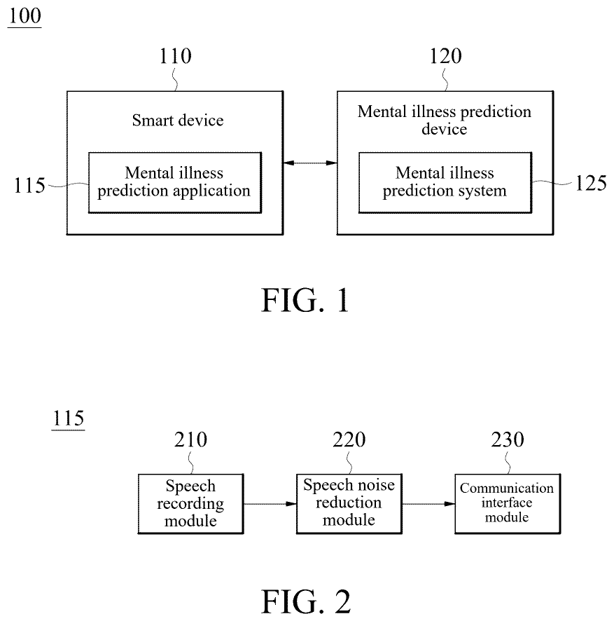

Referring to FIG. 1, FIG. 1 illustrates a block diagram of a system architecture of a mental health assessment system 100 according to an embodiment of the present invention. In the embodiment shown in FIG. 1, the mental health assessment system 100 includes a smart device 110 and a mental illness prediction device 120. The smart device 110 includes a mental illness prediction application 115. The mental illness prediction application 115 is installed in the smart device 110 and can be run via system resources of the smart device 110. The smart device 110 transmits inputted user's speech to the mental illness prediction device 120 to predict a probability of mental illness occurrence. The mental illness prediction device 120 may be a local server or a cloud server. The mental illness prediction device 120 includes a mental illness prediction system 125. The mental illness prediction device 120 provides resources, such as computing power, a memory, and a network, required for the operation of the mental illness prediction system 125. The mental illness prediction system 125 receives the user's speech transmitted by the smart device 110, analyzes and processes the user's speech to obtain a predicted value of the probability of mental illness occurrence, and then returns the predicted value to the mental illness prediction application 115. Data is transferred between the mental illness prediction application 115 and the mental illness prediction system 125 via application programming interface calls.

Referring to FIG. 2, FIG. 2 illustrates a block diagram of a system architecture of a mental illness prediction application 115 according to an embodiment of the present invention. In the embodiment shown in FIG. 2, the smart device 110 may be a smartphone, a tablet, a smart wearable device, or a smart mobile device. The smart device 110 can execute the mental illness prediction application 115. In an embodiment, the mental illness prediction application 115 includes: a speech recording module 210, a speech noise reduction module 220, and a communication interface module 230. The speech recording module 210 is configured to record a user's speech using the smart device 110. The speech noise reduction module 220 is configured to eliminate background noise from the user's speech using a noise reduction suite. The communication interface module 230 is configured to transmit the user's speech to a mental illness prediction system 125, and receive a probability of mental illness occurrence that is returned by the mental illness prediction system 125. In addition, the mental illness prediction application 115 further includes a function of executing a crisis intervention strategy and a function of generating a mental health assessment report.

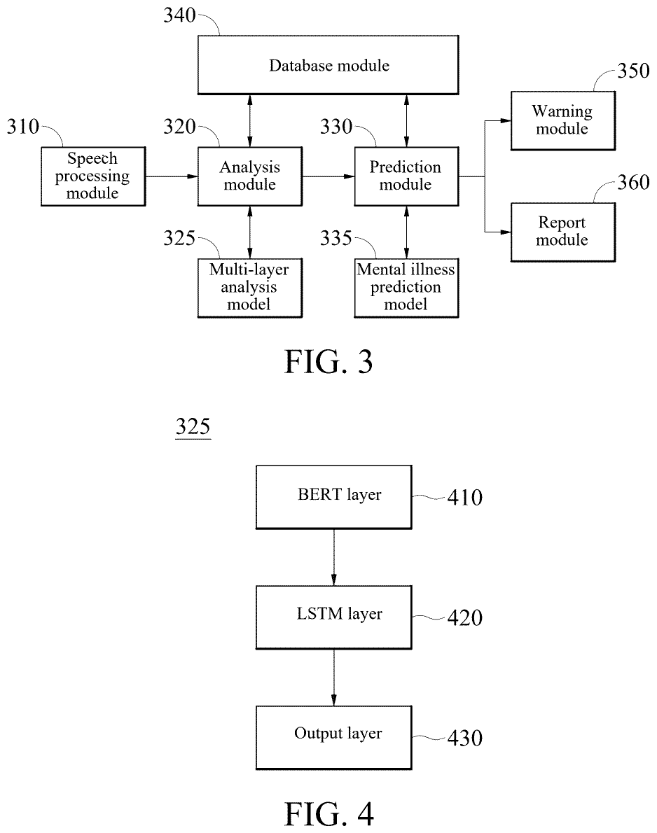

Referring to FIG. 3, FIG. 3 illustrates a block diagram of a first embodiment of a system architecture of a mental illness prediction system 125 according to an embodiment of the present invention. In the embodiment shown in FIG. 3, after the mental illness prediction device 120 receives the user's speech from the smart device 110, the user's speech will be transferred into the mental illness prediction system 125 to predict a probability of mental illness occurrence. The mental illness prediction system 125 includes a speech processing module 310, an analysis module 320, a multi-layer analysis model 325, a prediction module 330, a mental illness prediction model 335, a database module 340, a warning module 350, and a report module 360. The speech processing module 310 is configured to receive user's speech, and use a speech service application programming interface to convert the user's speech into a transcribed text. The analysis module 320 is configured to analyze the transcribed text using the multi-layer analysis model 325 to obtain a plurality of mental health indicators. The prediction module 330 is configured to infer the plurality of mental health indicators using the mental illness prediction model 335 to predict the probability of mental illness occurrence. The database module 340 is configured to store a plurality of knowledge bases using a cloud database, and store the mental health indicators and the probability of mental illness occurrence using the knowledge bases. In addition, the database module 340 further includes health knowledge stored in the cloud. The warning module 350 is configured to receive a mood score or a cognitive score obtained by standardizing the probability of mental illness occurrence, and determine whether the mood score is greater than an anxiety threshold or whether the cognitive score is less than a cognitive decline threshold, and execute a crisis intervention strategy when it is determined that the mood score is greater than the anxiety threshold or it is determined that the cognitive score is less than the cognitive decline threshold. The report module 360 is configured to receive the mood score or the cognitive score of each day in a specified date interval to draw a mood score curve graph or a cognitive score curve graph, and generate a mental health assessment report according to the mood score curve graph or the cognitive score curve graph.

In an embodiment, the speech processing module 310 is configured to convert user's speech into a transcribed text. The speech processing module 310 may be a speech transcription program suite, and the speech transcription program suite uses a speech recognition technology combined with a machine learning or deep learning model to interpret a signal of the user's speech and output a corresponding transcribed text. in addition, the speech processing module 310 may also be a speech service application programming interface of a cloud service platform, and the user's speech is converted into the transcribed text through the speech service application programming interface. The speech service application programming interface of the cloud service platform is, for example, Google Cloud Speech-to-Text, Amazon Web Services (AWS) Transcribe, and Microsoft Azure Speech Service.

Referring to FIG. 4, FIG. 4 illustrates a schematic diagram of multi-layer analysis model 325 of a mental illness prediction system 125 according to an embodiment of the present invention. In the embodiment shown in FIG. 4, the multi-layer analysis model 325 includes bidirectional encoder representations from transformers bidirectional encoder representations from transformers (BERTs) layer 410, a Long Short-Term Memory (LSTM) layer 420, and an output layer 430. The multi-layer analysis model 325 includes a tokenizer. The tokenizer is configured to segment the transcribed text into a plurality of tokens. The BERT layer 410 includes a BERT model. The BERT layer 410 is configured to generate a token embedded representation from a plurality of tokens using the BERT model. The LSTM layer 420 includes an LSTM model. The LSTM layer 420 is configured to embed tokens into the token embedded representation for time series analysis using the LSTM model to generate a sequence analysis output. The output layer 430 is configured to process the sequence analysis output using an activation function to generate an output value.

In an embodiment, the multi-layer analysis model 325 can use a tokenizer suitable for the BERT model to preprocess the transcribed text. For example, a BERT tokenizer in a Hugging Face transformers library is used to perform tokenization. The multi-layer analysis model 325 initializes the BERT tokenizer and BERT model with the Hugging Face transformers library, and uses a Bert-Base-Uncased pre-training model as the BERT model.

In an embodiment, the multi-layer analysis model 325 defines an LSTM layer 420 including a plurality (e.g., 50) of long short-term memory units. The LSTM layer 420 is a recurrent neural network (RNN) layer which is configured to process sequence data. In this embodiment, the LSTM layer 420 is configured to process an output from the BERT model.

In an embodiment, the multi-layer analysis model 325 uses a functional API of Keras to define a connection between different layers. The BERT layer 410 is connected to the LSTM layer 420 and the LSTM layer 420 is connected to the output layer 430 through the functional application programming interface of Keras, respectively. Specifically, in the functional application programming interface of Keras, the token embedded representation generated in the BERT layer 410 is used as an input of the LSTM layer 420, and the sequence analysis output generated in the LSTM layer 420 as an input of the output layer 430, respectively. This structure allows the output of the BERT layer 410 to be further processed by the LSTM layer 420 to capture more contextual information. In addition, the output of the LSTM layer 420 is connected to a Dense layer with a Sigmoid activation function for binary classification task.

In an embodiment, the multi-layer analysis model 325 is compiled using an Adam optimizer and a binary cross entropy loss function, and an accuracy rate is specified as an assessment indicator. The Adam optimizer is an efficient deep learning optimization algorithm, which is suitable for processing high-dimensional data and large-scale data sets. The binary cross entropy loss function is a loss function used for dealing with binary classification problems, which is suitable for classification tasks with output as a probability value. In the training process, as an assessment indicator, the accuracy rate is used for quantifying the classification performance of a model on a training data set or a test data set.

In an embodiment, the multi-layer analysis model 325 defines a prepare_data function for preprocessing the transcribed text. Firstly, tokenization is performed on the transcribed text with the prepare_data function, and then according to the input format requirements of the BERT model, padding is performed to convert the transcribed text into an input format acceptable to the BERT model. Finally, the prepare_data function returns a dictionary including input_ids and attention_masks. Input_ids is a digital code that corresponds to each token. attention_masks is a binary bit mask that corresponds to input_ids, and is used for indicating which positions are valid inputs and which positions are padded parts.

In an embodiment, the multi-layer analysis model 325 can transfer data preprocessed with the prepare_data function into the multi-layer analysis model 325 for analysis, inference, calculation, or prediction. For example, it used for prediction of emotional analysis.

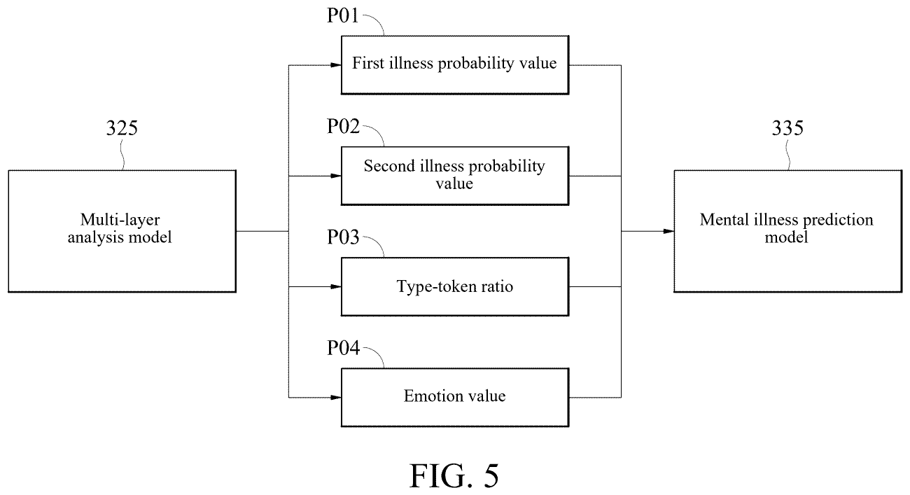

Referring to FIG. 5, FIG. 5 illustrates a schematic diagram of mental health indicators of a mental illness prediction system 125 according to an embodiment of the present invention. In the embodiment shown in FIG. 5, the multi-layer analysis model 325 analyzes the transcribed text to obtain a plurality of mental health indicators. The mental health indicator can be used as a reference for determining whether a user suffers from a mental illness.

In an embodiment, the multi-layer analysis model 325 analyzes an inputted transcribed text to obtain a speech rate, a number of pauses, and an average pause duration of the transcribed text; and infers according to the speech rate, the number of pauses, and the average pause duration of the transcribed text to predict a first illness probability value P01, and the first illness probability value P01 is used as a mental health indicator.

In an embodiment, the multi-layer analysis model 325 analyzes an inputted transcribed text to obtain a topic shift frequency and a sentence integrity proportion of the transcribed text; and infers according to the topic shift frequency and the sentence integrity proportion of the transcribed text to predict a second illness probability value P02, and the second illness probability value P02 is used as a mental health indicator.

In an embodiment, the multi-layer analysis model 325 analyzes an inputted transcribed text to obtain a number of token types of the transcribed text, that is, the number of unique tokens; and a number of tokens, which refers to a number of all tokens (tokens) in the text, including repeating tokens, i.e., a total number of tokens. The ratio of the number of unique tokens to the total number of tokens is calculated to obtain a type-token ratio (TTR) P03, and the type-token ratio P03 is used as the mental health indicator.

In an embodiment, the multi-layer analysis model 325 performs simulated prediction of emotional analysis on an inputted transcribed text to predict an emotion value P04, and the emotion value P04 is used as a mental health indicator.

In an embodiment, the multi-layer analysis model 325 is a multi-output ensemble model. The multi-layer analysis model 325 includes a first model, a second model, a third model, and a fourth model. The first model is configured to obtain the first illness probability value P01; the second model is configured to obtain the second illness probability value P02; the third model is configured to obtain the type-token ratio P03; and the fourth model is configured to obtain the emotion value P04.

In addition, in the embodiment shown in FIG. 5, the mental illness prediction model 335 infers the plurality of inputted mental health indicators to predict a probability of mental illness occurrence. The mental illness prediction model 335 may be constructed based on a logistic regression technology. Multiple variables are inputted into the mental illness prediction model 335 to predict a comprehensive probability value, and the comprehensive probability value is used as the probability of mental illness occurrence to predict a mental health status. Due to the characteristics of the logistic regression model, its predicted value has good interpretability, which can help professionals understand the impact of various variables on mental health, thereby providing a scientific basis for the prevention and intervention of mental illnesses. In addition, the mental illness prediction model 335 may also be constructed based on a model ensemble technology. The mental illness prediction model 335 receives a plurality of mental health indicators outputted from a plurality of models, and processes the mental health indicators by averaging, weighted averaging or voting to obtain a comprehensive probability value, thereby effectively reducing a possible deviation caused by a single model and thus improving the accuracy and robustness of prediction.

In addition, the mental illness prediction model 335 can integrate the multi-layer analysis model 325. The first illness probability value P01, the second illness probability value P02, the type-token ratio P03, and then emotion value P04 that are generated by the multi-layer analysis model 325 are transferred into the mental illness prediction model 335, and the comprehensive probability value is predicted according to the first illness probability value P01, the second illness probability value P02, the type-token ratio P03, and the emotion value P04, and the comprehensive probability value is used as the probability of mental illness occurrence.

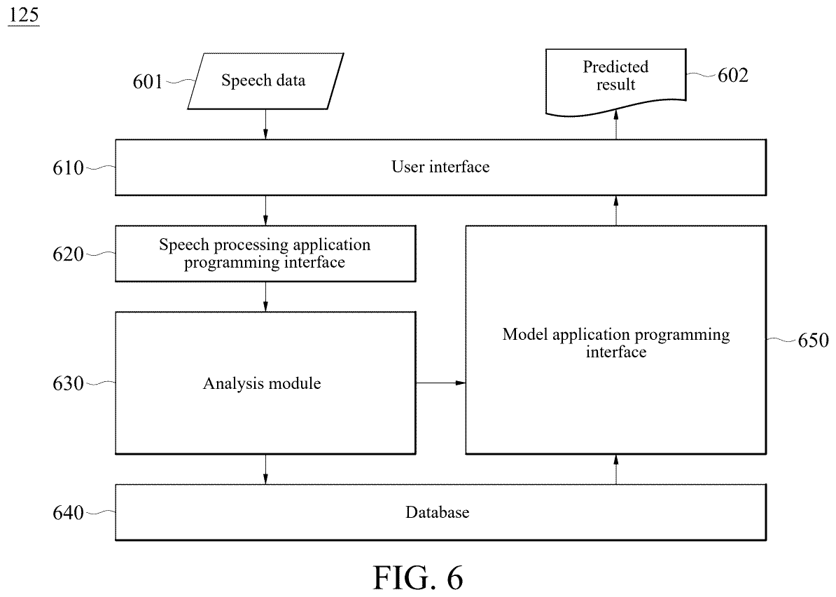

Referring to FIG. 6, FIG. 6 illustrates a schematic diagram of a second embodiment of a system architecture of a mental illness prediction system 125 according to an embodiment of the present invention. In the embodiment shown in FIG. 6, the mental illness prediction system 125 includes: a user interface 610, a speech processing application programming interface 620, an analysis model 630, a database 640, and a model application programming interface 650. In an embodiment, the mental illness prediction system 125 receives speech data 601 via the user interface 610. A user (or a target subject) records the speech data 601 of the target subject with a microphone, a recording device, or a smart device 110 (for example, a smartphone, a tablet, a smart speaker, a smart wearable device, a smart home assistant, or a medical device, etc.), and transfers the speech data 601 into the mental illness prediction system 125 via the user interface 610. The target subject may be populations at a high risk of dementia: age (over 65 years old), family history, genetic testing (APOE ε4, etc.), or cardiovascular history, or at a high risk of depression: populations with family history, life stress/traumatic experiences, mental illness history, or chronic illnesses. The content of the speech data 601 includes target subjects describing their feelings, symptoms, or their responses to health-related issues. A method for collecting the speech data 601 of the target subject through the smart device 110 includes: collecting specific situational conversations by active recording from a target subject, a caregiver or a doctor, for example, consulting room/online consultation process, on-site visit of the caregiver or online care records; and capturing daily conversations by device-triggered recording. In addition, before the mental illness prediction system 125 transmits the speech data 601 to the speech processing application programming interface 620, a noise reduction suite (for example, noisereduce) can be used for preprocessing the speech data 601, so as to eliminate background noise and other interference from the speech data 601 and improve the clarity of the speech data 601. The noise reduction suite may be installed in an application of the smart device or be installed in the mental illness prediction system 125 of a server.

In an embodiment, the speech processing application programming interface 620 may be regarded as one embodiment or a specific implementation of the speech service application programming interface. The mental illness prediction system 125 uses the speech processing application programming interface 620 to convert the speech data 601 into a transcribed text. The speech processing API 620 can be implemented through an Azure speech service API. The Azure speech service application programming interface is a speech transcription function provided by an Azure cloud service platform, which can accurately convert the speech data 601 into a transcribed text. Specifically, the Azure speech service application programming interface includes a speaker recognition function and a speech-to-text function. The speaker recognition function can identify the identity of the target subject with the speech data 601, and the speech-to-text can convert the speech data 601 into a transcribed text for in-depth natural language processing and analysis. The Azure speech service application programming interface provides a high-precision speech recognition function and can support multiple languages and dialects, thereby meeting the needs of a wide range of users. In this embodiment, although the Azure speech service application programming interface is taken as an example, it is also applicable to other cloud service platforms that include the speech processing application programming interface 620.

In an embodiment, the mental illness prediction system 125 is combined with the Azure speech service application programming interface and a large language model (LLM) technology of OpenAI to carry out in-depth analysis. The noise-reduced speech data 601 is transmitted to the Azure speech service application programming interface to perform speaker recognition and speech-to-text tasks. Firstly, an audio recognition sample of the target subject is registered via the speaker recognition function of the Azure speech service application programming interface, so as to establish a unique voice print model, thereby ensuring that the speech of the target subject can be accurately identified. Next, the speech of the target subject is transcribed in batches into a text using the speech-to-text function of the Azure speech service application programming interface. In addition, the mental illness prediction system 125 uses the speech processing application programming interface 620 to annotate the transcribed text with a timestamp. Each token is annotated with the timestamp in the transcribed text via the Azure speech service application programming interface; and the transcribed text is automatically segmented to generate complete sentences, and the start time and the end time of each sentence are marked.

In addition, the Azure speech service application programming interface and the large language model technology of OpenAI are utilized to assist in establishing the analysis model 630 of the mental illness prediction system 125, and its method includes the following steps:
1. Data integration: From an Azure cloud platform, importing medical data, including patient's medical records, laboratory test results, medical literature and the like. In addition, the text transcribed content of past consultation videos between a patient and a doctor is also imported, and these records can provide valuable contextual information to help the analysis model 630 better understand the semantics in the medical context.
2. Preprocessing: Cleaning and structuring the data, for example, removing duplicate records, standardizing terms and deleting irrelevant information. In addition, the text data is converted into a format readable by the analysis model 630 to ensure the accuracy and consistency of subsequent analysis.
3. Fine-tuning: Using an LLM technology of OpenAI to perform fine-tuning on the analysis model 630. In this process, the analysis model 630 will be adjusted according to the medical data and consultation records; and the analysis model 630 is trained to distinguish normal semantics from pathological semantics, thereby recognizing possible health issues or illness states more accurately.
4. Deep semantic analysis: Using the fine-tuned analysis model 630 to carry out semantic analysis to recognize key information in the text, such as symptoms, illnesses and treatment suggestions. Meanwhile, the deep learning ability of the analysis model 630 is used for carrying out emotional analysis on the text to identify the emotional state of the patient, for example, anxiety, depression and the like.

A test sample of the analysis model 630 is 5-minute excerpts of conversations between the target subject and his family, including the following fields: utterance start time, utterance end time, speaker, utterance content, and token timestamp (the time of each token in the utterance). The test sample is converted into a transcribed text via the speech processing application programming interface 620. The analysis model 630 includes a plurality of models and processes the transcribed text of the test sample with the plurality of models. In an embodiment, the analysis model 630 includes a model for analyzing speech rate and the number of pauses, a model for analyzing semantic coherence, a model for analyzing sentence integrity proportion, a model for analyzing token richness, and a model for analyzing emotional expression. The above-mentioned all models can be constructed based on the BERT model and/or the LSTM model. In addition, the analysis model 630 may also include a logistic regression model.

In an embodiment, the analysis model 630 carries out time series analysis through an LSTM model, predicts the risk of developing a mental illness according to the speech rate, the number of pauses, and the average pause duration of the test sample, and outputs a predicted score between 0 and 1. The variables are described as follows:
1. Speech rate: Calculating the number of tokens per minute in a speech and recording the daily average number of tokens per minute. The number of tokens per minute is calculated from the start time, end time, and the number of tokens of each sentence in a speech-to-text result file (or the transcribed text of the test sample), and then all data on the same day is summarized to calculate an average value.
2. Number of pauses: Recognizing blank paragraphs in sentences and counting the number of pauses in daily speech. According to the speech-to-text result file (or the transcribed text of the test sample), a time difference between the pronunciation timestamp of each token and the pronunciation timestamp of a next token is calculated. If the time interval is greater than or equal to 0.5 seconds, it is determined that there is a pause, and finally all the pauses in the same day are summarized.
3. Average pause duration: Recognizing the blank paragraphs in sentences and calculating the average duration of daily pauses. According to the speech-to-text result file (or the transcribed text of the test sample), the pronunciation timestamp of each token in each sentence is compared with the pronunciation timestamp of a next token to calculate a time difference, and then the time differences of all the pauses are counted and the average pause duration in the same day is calculated.

In an embodiment, it is calculated that the speech rate of the test sample is 90 tokens per minute, the number of pauses is 8, and the average pause time is 0.6 seconds. The established LSTM model is used to predict the first illness probability value P01 according to the speech rate, the number of pauses and the average pause time, and it is derived that the first illness probability value P01 is 0.78, and the first illness probability value P01 is used as the mental health indicator.

In an embodiment, the analysis model 630 carries out time series analysis through an LSTM model, predicts the risk of developing a mental illness according to the topic shift frequency, and the sentence integrity proportion of the test sample, and outputs a predicted score between 0 and 1. The variables are described as follows:
1. topic shift frequency: Using a large language model (LLM) to recognize key tokens or phrases in the text, where these tokens are usually related to specific topics, and recognizing the changes of topics in the text. When the content of adjacent texts is inconsistent in topic classification, it is marked as topic shift. The frequency of daily topic shift is calculated. Currently, topics have been classified regarding daily life, learning feelings, social activities, personal feelings and the like. The table below shows the results of classification for topics in test samples.

| Content | Classification |
|---|---|
| Hmm, it's just like that, nothing special. | Daily feeling |
| It's all the same to me, and I find | Daily feeling |
| everything is pretty boring. | |
| No, I don't really want to go out. | Social activities |
| I don't know, I just feel very tired and | Personal feeling |
| not happy at all. | |
| Thanks, mom, I'll think about it. | Daily feeling |

The test sample includes three topic shifts, and thus, the topic shift frequency is 0.6
2. Sentence integrity proportion: Assessing the proportion of complete sentences to the total number of sentences in a speech. A lower proportion may indicate problems in grammatical structure and language expression. The daily average sentence integrity proportion is calculated. The BERT model (for example, a Bert-Base-Chinese pre-training model) in Hugging Face transformers library is used to assess sentence integrity. Firstly, the BERT model is trained using complete sentence and incomplete sentence data, and then the established BERT model is used to predict the test samples. The table below shows the results of classification for sentence integrity in test samples.

| Content | Classification |
|---|---|
| Hmm, it's just like that, nothing special. | Incomplete sentence |
| It's all the same to me, and I find | Incomplete sentence |
| everything is pretty boring. | |
| No, I don't really want to go out. | Complete sentence |
| I don't know, I just feel very tired and | Complete sentence |
| not happy at all. | |
| Thanks, mom, I'll think about it. | Incomplete sentence |

The test sample includes 2 complete sentences, and thus, the sentence integrity proportion is 0.4.

In an embodiment, the topic shift frequency of the test sample is 0.6, and the sentence integrity proportion is 0.4, and the established LSTM model is used to predict the second illness probability value P02 according to the topic shift frequency and the sentence integrity proportion, and it is derived that the second illness probability value P02 is 0.95, and the second illness probability value P02 is used as the mental health indicator.

In an embodiment, the analysis model 630 calculates the type-token ratio P03 based on the ratio of the number of unique tokens to the total number of tokens in the test sample, and utilizes the type-token ratio P03 to assess the degree of token diversity, and the steps are as follows:
1. Tokenization: splitting a text into tokens. This typically involves segmenting the text according to spaces, punctuation marks, etc., to split the text into the tokens.
2. Calculation of number of token types: This is the number of unique tokens in the text.
3. Calculation of number of tokens: This is the total number of all tokens (including repetitions) in the text.
4. Calculation of ratio of number of unique tokens to total number of tokens: This ratio is the type-token ratio P03 ranging from 0 to 1. The closer the value is to 1, the richer and more diverse the tokens are. Conversely, the lower the ratio, the more obvious the repeated use of tokens.

In an embodiment, the analysis module 320 uses the BERT tokenizer in the Hugging Face transformers library to perform tokenization and it is calculated that the type-token ratio P03 of the test sample is 0.61, and the type-token ratio P03 is used as the mental health indicator.

In an embodiment, the analysis model 630 uses the powerful contextual understanding ability of BERT model and the time series data processing ability of LSTM model to improve the understanding ability to long texts and conversations, and can capture emotional fluctuations and semantic changes more accurately. Firstly, starting from the preprocessing of an original text, which includes converting the text into a format that BERT model can process, for example, performing tokenization and adding special tags. Next, we use the pre-trained BERT model to extract the embedded representation of each token in the text, which enables the analysis model 630 to deeply understand the meaning of tokens in a specific context and provide more accurate emotional and semantic analysis. Then, these embedded representations are used as the input of the LSTM mode. The LSTM model is specially configured to process time series data, so that the model can capture and analyze the time series correlation in long texts or conversations. Finally, an emotion value P04 between 0 and 1 is generated, which represents negative to positive intensity.

In an embodiment, the output results of emotional analysis on the test samples by the analysis model 630 are as follows:
1. Text: “Hmm, it's just like that, nothing special. It's all the same to me, and I find everything is pretty boring. No, I don't really want to go out. I don't know, I just feel tired and not happy at all. Thanks, mom, I'll think about it.”
2. Predicted mood: Low.
3. Predicted probability value: 0.3282.

In addition, the mental illness prediction system 125 can import public datasets in a Kaggle platform to optimize the analysis model 630. The public dataset of the Kaggle platform includes customer reviews for six American airlines and is divided into negative, neutral, and positive. An original two-category (negative and positive) model is adjusted as a three-category model, and model establishment and prediction are performed to accurately quantify the emotion value P04. Since most of the public datasets in the Kaggle platform are in English, firstly, a large-scale language model can be used to translate data into Chinese, with an instruction to use vocabulary closer to daily life. The table below shows the output results of emotional analysis of test samples by analysis model 630.

| | | Probability |
|---|---|---|
| Content | Classification | % |
| Hmm, it's just like that, nothing | Negative | 14.5 |
| special. | | |
| It's all the same to me, and I find | Negative | 2.40 |
| everything is pretty boring. | | |
| No, I don't really want to go out. | Neutral | 62.0 |
| I don't know, I just feel very tired | Negative | 3.30 |
| and not happy at all. | | |
| Thanks, mom, I'll think about it. | Negative | 3.70 |

In an embodiment, the analysis model 630 (e.g., the logistic regression model) carries out comprehensive analysis based on a plurality of input variables to generate a comprehensive probability value, and the comprehensive probability value is taken as the probability of mental illness occurrence. The analysis model 630 can be trained as a depression mood determination model or a dementia cognitive determination model by adjusting parameters according to the plurality of input variables. The plurality of variables include: the first illness probability value P01 generated according to the speech rate, the number of pauses and the average pause duration; the second illness probability value P02 generated according to the topic shift frequency and the sentence integrity proportion; the type-token ratio P03; and the emotion value P04.

The mental illness prediction system 125 includes a model application programming interface 650, for example, a depression model application programming interface and a dementia model application programming interface. The model application programming interface 650 encapsulates the analysis model 630 as an application programming interface and deploys the application programming interface to a server or cloud service platform. A user or application sends a request to the analysis model 630 via the model application programming interface 650, and returns a predicted result 602 of the analysis model 630 to a requester in a standardized format. The predicted result 602 can be returned to the mental illness prediction application 115 of the smart device 110 via the user interface 610. In addition, the analysis model 630 established for analyses such as language emotional analysis, positive and negative nature determination of topics, speech rate and intonation, semantic coherence, usage rate of repeating tokens, speech rate and pause time may also be encapsulated as the model application programming interface 650.

The mental illness prediction system 125 includes a database 640. The database 640 is configured to store the test samples and the predicted results 602 of the analysis model 630 (for example, mental health indicators and the probability of mental illness occurrence). The database 640 may employ a PostgreSQL database management system or other cloud database systems. In addition, the mental illness prediction system 125 includes a knowledge base. The knowledge base is configured to store health knowledge for comparison with the predicted results 602 generated by the analysis model 630. The knowledge base may employ cloud object storage services provided by Azure, for example, Azure blob storage.

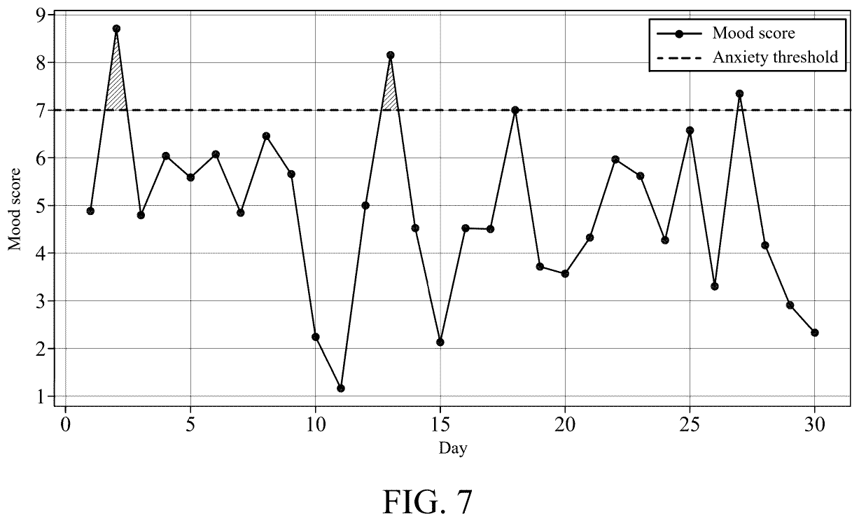

Referring to FIG. 7, FIG. 7 illustrates a curve graph of mood scores of a depression mood determination model according to an embodiment of the present invention. In the embodiment shown in FIG. 7, the mental illness prediction model 335 or the analysis model 630 (e.g., the logistic regression model) of the mental illness prediction system 125 can be trained into a depression mood determination model to predict the mood score. Factors that affect the mood score include: the emotion of language, positive and negative nature of topics, and speech rate and intonation, and the like. The mental illness prediction model 335 or the analysis model 630 conducts comprehensive analysis based on a plurality of inputted variables to generate a comprehensive probability value, and standardizes the comprehensive probability value into a mood score with a score ranging from 1 to 10. In addition, the report module 360 of the mental illness prediction system 125 draws a mood score curve graph according to the received mood score of each day within a specified date interval, and generates a mental health assessment report according to the mood score curve graph. FIG. 7 shows mood scores of each day of a target subject within 30 days, mainly used for determining whether the daily mood score is greater than an anxiety threshold. In an embodiment, the anxiety threshold is set as 7.

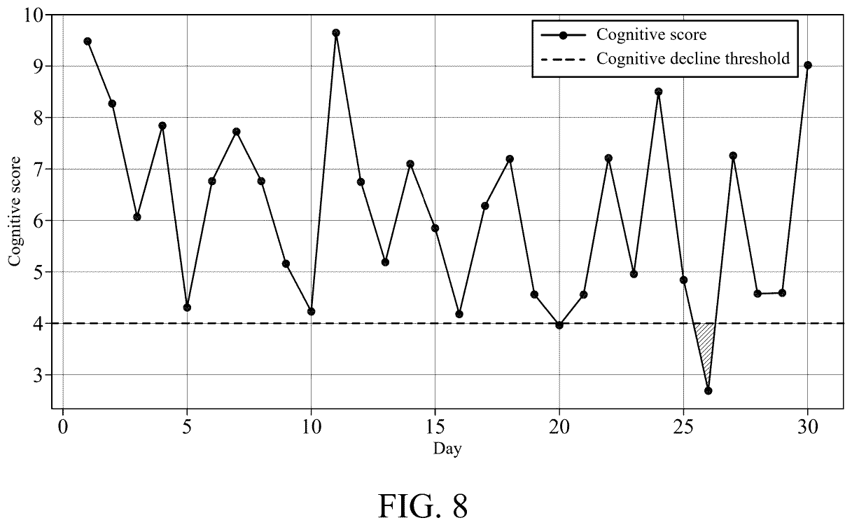

Referring to FIG. 8, FIG. 8 illustrates a curve graph of cognitive scores of a dementia cognitive determination model according to an embodiment of the present invention. In the embodiment shown in FIG. 8, the mental illness prediction model 335 or the analysis model 630 (e.g., the logistic regression model) of the mental illness prediction system 125 can be trained into a dementia cognitive determination model to predict the cognitive score. Factors that affect the cognitive score include: semantic coherence, the use of repeating tokens, memory signs, speech rate and pause patterns, and the like. The mental illness prediction model 335 or the analysis model 630 conducts comprehensive analysis based on a plurality of inputted variables to generate a comprehensive probability value, and standardizes the comprehensive probability value into a cognitive score with a score ranging from 1 to 10. In addition, the report module 360 of the mental illness prediction system 125 draws a cognitive score curve graph according to the received cognitive scores of each day within a specified date interval, and generates a mental health assessment report according to the cognitive score curve graph. FIG. 8 shows cognitive scores of each day of a target subject within 30 days, mainly used for determining whether the daily cognitive score is less than a cognitive decline threshold. In an embodiment, the cognitive decline threshold is set as 4.

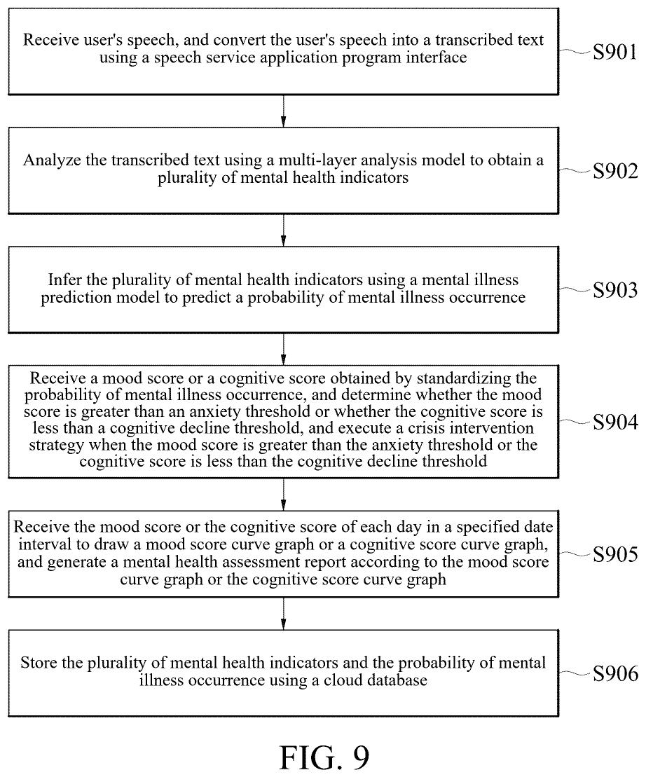

Referring to FIG. 9, FIG. 9 illustrates a flowchart of a mental illness prediction method according to an embodiment of the present invention. In the embodiment shown in FIG. 9, the mental illness prediction method includes: receiving user's speech, and converting the user's speech into a transcribed text using a speech service application programming interface (step S901); analyzing the transcribed text using a multi-layer analysis model 325 to obtain a plurality of mental health indicators (step S902); inferring the plurality of mental health indicators using a mental illness prediction model 335 to predict a probability of mental illness occurrence (step S903); receiving a mood score or a cognitive score obtained by standardizing the probability of mental illness occurrence, and determining whether the mood score is greater than an anxiety threshold or determining whether the cognitive score is less than a cognitive decline threshold, and executing a crisis intervention strategy when the mood score is greater than the anxiety threshold or the cognitive score is less than the cognitive decline threshold (step S904); receiving the mood score or the cognitive score of each day in a specified date interval to draw a mood score curve graph or a cognitive score curve graph, and generating a mental health assessment report according to the mood score curve graph or the cognitive score curve graph (step S905); and storing the plurality of mental health indicators and the probability of mental illness occurrence using a cloud database (step S906).

Although the present invention has been described in considerable detail with reference to certain preferred embodiments thereof, the disclosure is not for limiting the scope of the invention. Persons having ordinary skill in the art may make various modifications and changes without departing from the scope and spirit of the invention. Therefore, the scope of the appended claims should not be limited to the description of the preferred embodiments described above.

What is claimed is:

1. A mental illness prediction method, comprising:
receiving user's speech, and converting the user's speech into a transcribed text using a speech service application programming interface (API);
analyzing the transcribed text using a multi-layer analysis model to obtain a plurality of mental health indicators; and
inferring the plurality of mental health indicators using a mental illness prediction model to predict a probability of mental illness occurrence.

2. The mental illness prediction method according to claim 1, wherein the multi-layer analysis model uses a tokenizer to segment the transcribed text into a plurality of tokens.

3. The mental illness prediction method according to claim 2, wherein the multi-layer analysis model comprises a BERT layer, an LSTM layer, and an output layer, the BERT layer comprising a BERT model, and the BERT layer using the BERT model to generate a token embedded representation from the plurality of tokens; the LSTM layer comprising an LSTM model, and the LSTM layer using the LSTM model to carry out time series analysis on the token embedded representation to generate a sequence analysis output; and the output layer using an activation function to process the sequence analysis output to generate an output value, and using the output value as a mental health indicator.

4. The mental illness prediction method according to claim 3, wherein:
the multi-layer analysis model uses a functional application programming interface of Keras to connect the BERT layer to the LSTM layer and connect the LSTM layer to the output layer, respectively; and
the multi-layer analysis model is compiled using an Adam optimizer and a binary cross entropy loss function, and an accuracy rate is specified as an assessment indicator.

5. The mental illness prediction method according to claim 1, wherein the multi-layer analysis model analyzes the transcribed text to obtain a speech rate, a number of pauses and an average pause duration of the transcribed text, and infers according to the speech rate, the number of pauses and the average pause duration of the transcribed text to predict a first illness probability value, and the first illness probability value is used as the mental health indicator.

6. The mental illness prediction method according to claim 1, wherein the multi-layer analysis model analyzes the transcribed text to obtain a topic shift frequency and a sentence integrity proportion of the transcribed text, and infers according to the topic shift frequency and the sentence integrity proportion of the transcribed text to predict a second illness probability value, and the second illness probability value is used as the mental health indicator.

7. The mental illness prediction method according to claim 1, wherein the multi-layer analysis model analyzes the transcribed text to obtain a number of unique tokens and a total number of tokens of the transcribed text, and calculates a ratio of the number of unique tokens to the total number of tokens of the transcribed text to obtain a type-token ratio, and the type-token ratio is used as the mental health indicator.

8. The mental illness prediction method according to claim 1, wherein the multi-layer analysis model makes an emotional analysis inference according to the transcribed text to predict an emotion value, and the emotion value is used as the mental health indicator.

9. The mental illness prediction method according to claim 8, wherein the multi-layer analysis model is trained using a public dataset from Kaggle, and classifies the transcribed text into three categories of positive, neutral, and negative to accurately quantify the emotion value.

10. The mental illness prediction method according to claim 1, wherein the mental illness prediction model is established based on a logistic regression model.

11. The mental illness prediction method according to claim 10, wherein the mental illness prediction model receives a first illness probability value, a second illness probability value, a type-token ratio, and an emotion value that are generated by the multi-layer analysis model, and predicts a comprehensive probability value according to the first illness probability value, the second illness probability value, the type-token ratio, and the emotion value, and the comprehensive probability value is used as the probability of mental illness occurrence.

12. The mental illness prediction method according to claim 11, wherein the mental illness prediction model is a depression mood determination model, and the comprehensive probability value is standardized to a first standardized score, and the first standardized score is used as a mood score.

13. The mental illness prediction method according to claim 11, wherein the mental illness prediction model is a dementia cognitive determination model, and the comprehensive probability value is standardized to a second standardized score, and the second standardized score is used as a cognitive score.

14. The mental illness prediction method according to claim 1, further comprising:
receiving a mood score or a cognitive score obtained by standardizing the probability of mental illness occurrence, and determining whether the mood score is greater than an anxiety threshold or whether the cognitive score is less than a cognitive decline threshold, and executing a crisis intervention strategy when the mood score is greater than the anxiety threshold or the cognitive score is less than the cognitive decline threshold;
receiving the mood score or the cognitive score of each day in a specified date interval to draw a mood score curve graph or a cognitive score curve graph, and generating a mental health assessment report according to the mood score curve graph or the cognitive score curve graph; and
storing the plurality of mental health indicators and the probability of mental illness occurrence using a cloud database.

15. A mental illness prediction device, comprising:
a mental illness prediction system, configured to predict a probability of mental illness occurrence, and comprising:
a speech processing module, configured to receive user's speech, and convert the user's speech into a transcribed text using a speech service application programming interface (API);
an analysis module, configured to analyze the transcribed text using a multi-layer analysis model to obtain a plurality of mental health indicators; and
a prediction module, configured to infer the plurality of mental health indicators using a mental illness prediction model to predict a probability of mental illness occurrence.

16. The mental illness prediction device according to claim 15, wherein the multi-layer analysis model is configured to:
analyze the transcribed text to obtain a speech rate, a number of pauses, and average pause duration, and predict a first illness probability value;
analyze the transcribed text to obtain a topic shift frequency, a sentence integrity proportion, and predict a second illness probability value;
analyze the transcribed text to obtain a number of unique tokens and a total number of tokens, and calculate a type-token ratio; and
carry out an emotional analysis according to the transcribed text to predict an emotion value,
wherein the first illness probability value, the second illness probability value, the type-token ratio and the emotion value are used as the plurality of mental health indicators.

17. The mental illness prediction device according to claim 15, wherein the mental illness prediction system further comprises:
a database module, configured to store the plurality of mental health indicators and the probability of mental illness occurrence using a cloud database;
a warning module, configured to receive a mood score or a cognitive score obtained by standardizing, through the prediction module, the probability of mental illness occurrence, and execute a crisis intervention strategy when it is determined that the mood score is greater than an anxiety threshold or it is determined that the cognitive score is less than a cognitive decline threshold; and
a report module, configured to receive the mood score or the cognitive score of each day in a specified date interval to draw a mood score curve graph or a cognitive score curve graph, and generate a mental health assessment report according to the mood score curve graph or the cognitive score curve graph.

18. A mental illness prediction application, applied to a smart device, and comprising:
a speech recording module, configured to record user's speech using the smart device; and
a communication interface module, configured to transmit the user's speech to a mental illness prediction system, and receive a probability of mental illness occurrence that is returned by the mental illness prediction system.

19. The mental illness prediction application according to claim 18, further comprising:
a speech noise reduction module, configured to eliminate background noise from the user's speech using a noise reduction suite.

20. The mental illness prediction application according to claim 19, wherein the mental illness prediction system is configured to:
convert the user's speech into a transcribed text using a speech service application programming interface (API);
analyze the transcribed text using a multi-layer analysis model to obtain a plurality of mental health indicators; and
infer the plurality of mental health indicators using a mental illness prediction model to predict the probability of mental illness occurrence.
